(19) United States
(12) Patent Application Publication
(10) Pub. No.: US 2012/0071415 A1
(43) Pub. Date: Mar. 22, 2012

(54) DOMINANT NEGATIVE MUTANTS OF SAM68 FOR USE IN THE TREATMENT OF SPINAL MUSCULAR ATROPHY (SMA)

(75) Inventors: Maria Paola Paronetto, Roma (IT); Simona Pedrotti, Roma (IT)

(73) Assignee: FONDAZIONE SANTA LUCIA, Roma (IT)

(21) Appl. No.: 13/145,629

(22) PCT Filed: Jan. 20, 2010
(86) PCT No.: PCT/EP2010/050650

§ 371 Date: Dec. 8, 2011

(30) Foreign Application Priority Data
Jan. 21, 2009 (IT) RM2009A 000021

(51) Int. Cl. A61K 38/17; A61P 21/00; A61K 48/00; A61P 25/00; C12N 15/63; C07K 14/47

(52) U.S. Cl. 514/17.7; 435/320.1; 530/350; 514/44 R

(57) ABSTRACT

The present invention relates to the use of dominant negative mutants of Sam68 for the manufacture of a medicament for the treatment of spinal muscular atrophy, to nucleic acids coding for such mutants and to vectors and methods related thereto.

The present invention relates to the use of dominant negative mutants of Sam68 for the manufacture of a medicament for the treatment of spinal muscular atrophy, to nucleic acids coding for such mutants and to vectors and methods related thereto.

### STATE OF THE ART

Spinal Muscular Atrophy (hereinafter also referred to as SMA) is an autosomal recessive neuromuscular disorder that represents the primary genetic cause of infant mortality, with an incidence of 1 in 6000 in the human population. SMA can be classified in three types based on disease severity, with type I being the most severe and type III the mildest form (Zerres and Rudnik-Schonenberg, 1995). SMA is characterized by the degeneration of motor neurons in the anterior horn of the spinal cord and by consequent skeletal muscle atrophy (Monani, 2005). The genetic cause of SMA is a homozygous loss of SMN1, a gene located in the telomeric region of chromosome 5 that encodes the “survival motor neuron” protein (hereinafter also referred to as SMN protein or SMN). Notably, all SMA patients retain at least one copy of the centromeric and almost identical SMN2 gene. Although SMN2 encodes a virtually identical protein, the expression levels of this gene are however not sufficient to restore SMN activity (Monani, 2005). The instability of SMN2 protein derives from a single substitution, a C to T at position +6 in exon 7, which is translationally silent but causes the skipping of this exon in most of the SMN2 transcripts (Lorson et al., 1999; Monani et al., 1999). The resulting protein is highly unstable and does not support survival and function of spinal α-motoneurons, thereby causing the disease. For this reason, the regulation of exon 7 alternative splicing in the SMN2 mRNA represents a clinically important model to investigate the impact of splicing regulation in human pathologies (Cartegni et al., 2002; Pellizzoni, 2007; Wang and Cooper, 2007). Two models have been proposed to explain the effect caused by the C-to-T substitution in SMN2 exon 7. Cartegni and Krainer (2002) have proposed that this transition disrupts an exonic splicing enhancer (ESE) and impairs the binding of the splicing factor ASF/SF2, thereby affecting exon recognition. By contrast, the alternative model proposes that this single nucleotide change creates an exonic splicing silencer (ESS) to which the splicing repressor hnRNP A1 binds, thereby favouring exon 7 skipping from the SMN2 pre-mRNA (Kashima and Manley, 2003). Further support to this latter model was provided by the observation that hnRNP Al, but not ASF/SF2, interacted strongly and specifically with SMN2 exon 7 and that its effect on exon skipping was highly specific (Kashima et al., 2007). A positive regulator of exon 7 inclusion playing an hnRNP A1-antagonistic role is the splicing factor TRA2β (Hofmann et al., 2000; Chang et al., 2001), indicating that the relative expression levels of specific splicing factors can strongly affect alternative splicing of SMN2 pre-mRNA.

In some individuals affected by SMA, the SMN2 gene may be replicated up to four times and the presence of additional SMN2 genes can help replace the protein needed for the survival of motor neurons. As a result, individuals with more copies of this gene experience less severe symptoms.

As there is currently no cure for SMA and its treatment only focuses on the management of symptoms and is still scarcely effective, the need is felt to find medicaments for the treatment of this disorder.

### SUMMARY OF THE INVENTION

It is an object of the present invention to provide the use of a dominant negative mutant of SEQ ID NO:1 for the manufacture of a medicament for the treatment of SMA.

It is another object of the present invention to provide the use of the dominant negative mutant of SEQ ID NO:1 for the manufacture of a medicament for the treatment of SMA so that survival motor neuron (SMN) protein expression is rescued in the cells of an individual affected by SMA.

It is a further object of the present invention to provide a vector for gene therapy including a nucleic acid encoding for a dominant negative mutant of SEQ ID NO:1.

It is a further object of the present invention to provide a dominant negative mutant of SEQ ID NO:1 for use in the treatment of SMA.

Finally, it is an object of the present invention to provide a method for rescuing survival motor neuron (SMN) protein expression in the cells of an individual affected by spinal muscular atrophy for the treatment of SMA comprising administering a dominant negative mutant Sam68 polypeptide and/or nucleic acid to said cells.

### Definitions

As used herein, the term “dominant negative mutant” of a protein refers to a mutant polypeptide or nucleic acid, which lacks wild-type activity and which, once expressed in a cell wherein a wild-type of the same protein is also expressed, dominates the wild-type protein and effectively competes with wild type proteins for substrates, ligands, etc., and thereby inhibits the activity of the wild type molecule.

In particular, the term “mutant polypeptide” is intended to include any polypeptide or representation thereof that differs from its corresponding wild type polypeptide by having at least one amino acid substitution, addition or deletion, for example a glutamine addition, preferably it consists of an amino acid substitution.

Advantageously, the preferred mutant polypeptides according to the present invention differ from their corresponding wild type polypeptide by having one or two amino acid substitution or by presenting the deletion of N-terminal comprising the GSG domain.

The term “GSG domain” refers to a highly conserved region (GRP33/Sam68/GLD1) which is required for homodimerization and sequence specific RNA binding.

As used herein, the term “Sam68” refers to the protein of SEQ ID NO:1.

As used herein, the term “Sam68_{V229F}” refers to the protein of SEQ ID NO:2.

As used herein, the term “Sam68_{NLS-KO}” refers to the protein of SEQ ID NO:3.

As used herein, the term “Sam68₃₅₁₋₄₄₃” refers to the protein of SEQ ID NO:4.

As used herein, the term “Sam68-DNA” refers to the DNA of SEQ ID NO:5.

As used herein, the term “Sam68_{V229F}-DNA” refers to the DNA of SEQ ID NO:6.

As used herein, the term “Sam68_{NLS-KO}-DNA” refers to the DNA of SEQ ID NO:7.

As used herein, the term “Sam68₃₅₁₋₄₄₃-DNA” refers to the DNA of SEQ ID NO:8.

### BRIEF DESCRIPTION OF THE DRAWINGS

The present invention will now be described with reference to the accompanying drawings, wherein:

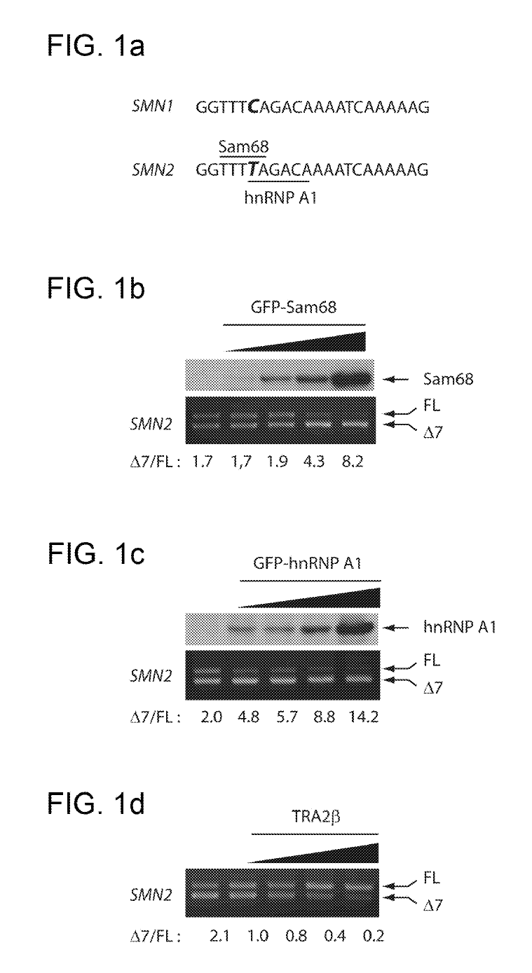

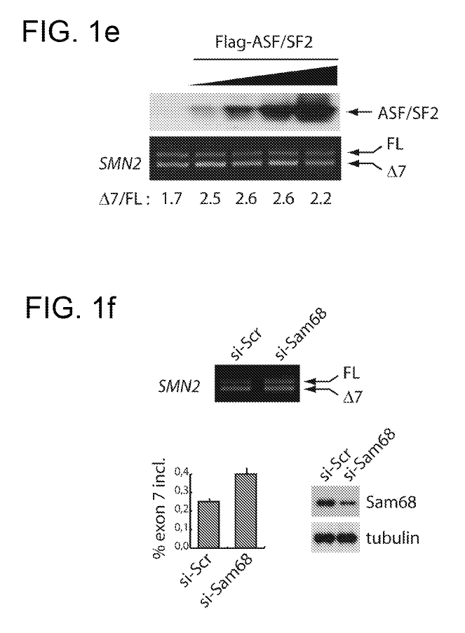

FIG. 1 shows the results relating to the induction by Sam68 of exon 7 skipping in the SMN2 pre-mRNA.
(A) The SMN1 and SMN2 exon 7 sequences are schematically represented and the C to T transition is highlighted in a bold character. The putative binding sites for Sam68 and hnRNP A1 in SMN2 exon 7 are indicated. (B-E) Splicing assay were performed by cotransfecting 0.5 μg of the pCI-SMN2 minigene and increasing amounts of GFP-Sam68 (B), GFP-hnRNP A1 (C), pCDNA3-Tra2β (D), Flag-ASF/SF2 (E) or si-Sam68 dsRNAs or si-Scrambled dsRNAs (F) in HEK293T cells. Cells were harvested 24 hours after transfection and 1 μg of total RNA was used in RT-PCR experiments. Western blot analyses were performed for each experiment. Densitometric analysis of the ratio between Δexon7/full length SMN2 was performed using ImageQuant5.

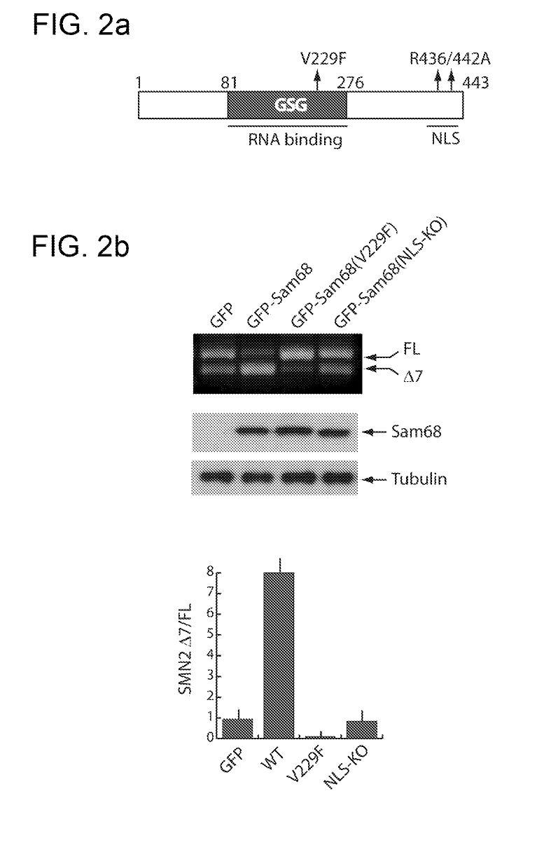

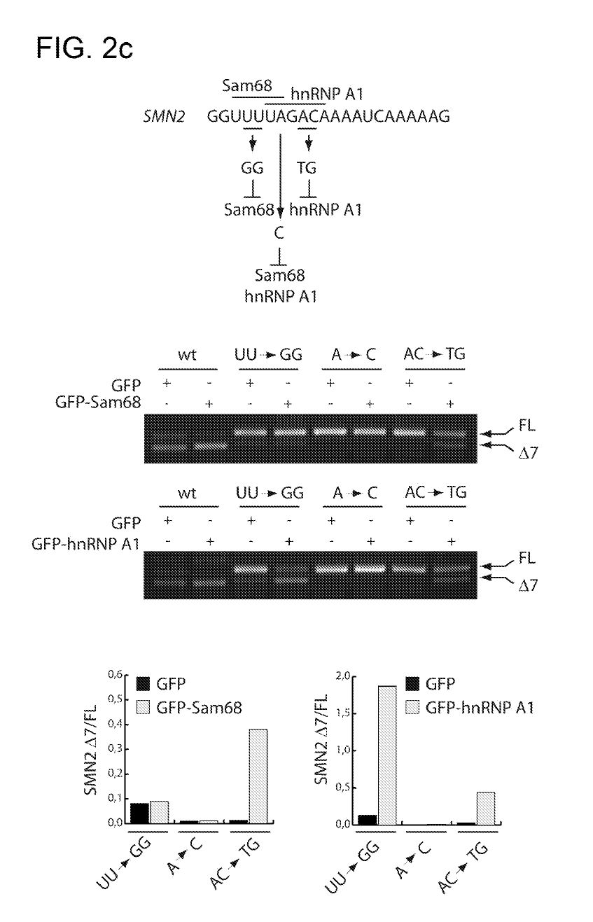

FIG. 2 shows the results relating to the requirement of the binding of Sam68 to SMN2 mRNA for exon 7 skipping.
(A) Schematic diagram representing the STAR (signal transduction and activation of RNA) protein Sam68 and the mutations introduced in the RNA binding domain (V229F) and nuclear localization signal (NLS; R436/442A). (B) Splicing assay of the SMN2 minigene in HEK293T cells cotransfected with the indicated constructs. Cells were harvested 24 hours after transfection and processed for RT-PCR experiments (top panel). Cells extract from the same sample were analyzed by Western blot (bottom panel) for GFP (top) and tubulin (bottom) as loading control. Densitometric analysis of the RT-PCR experiments is shown below. (C) Schematic diagram of SMN2 exon 7 indicating the mutations introduced in the putative binding sites for Sam68 and hnRNP A1. RT-PCR analysis of the splicing assays in the presence or absence of transfected GFP-Sam68 (upper panel) or GFP-hnRNP A1 (lower panel) are shown. Densitometric analysis is shown in the bar graphs.

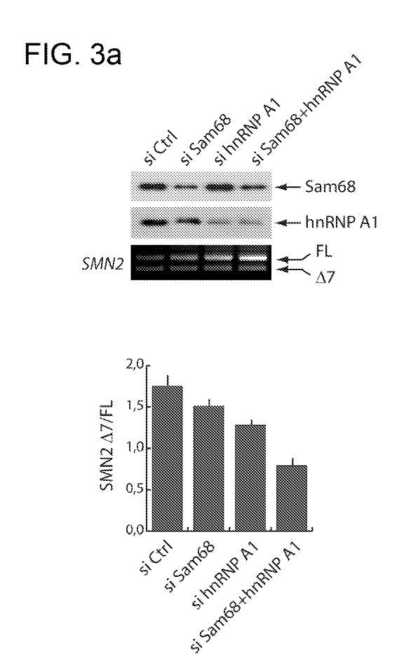

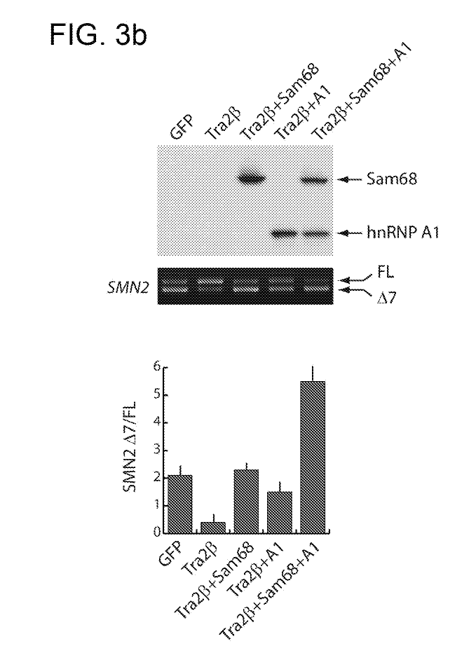

FIG. 3 shows cooperation of Sam68 and hnRNP A1 in SMN2 exon 7 skipping.
(A) HEK293T cells were transfected with scrambled, Sam68 or hnRNP A1 siRNA either alone or in combination. After 24 hours, cells were transfected with pCI-SMN2 minigene and analysed by RT-PCR for alternative splicing. Densitometric analysis of the splicing assay is shown below. Western blot analisys for Sam68 and hnRNP A1 is shown above the PCR analysis. (B) HEK293T cells were transfected with pCI-SMN2 and plasmids encoding TRA2β Sam68 or hnRNP A1 either alone or in combination. After 24 hours, cells were analysed by RT-PCR for alternative splicing. Densitometric analysis of the splicing assay is shown below. Western blot analysis for TRA2β, Sam68 and hnRNP A1 is shown above the PCR analysis.

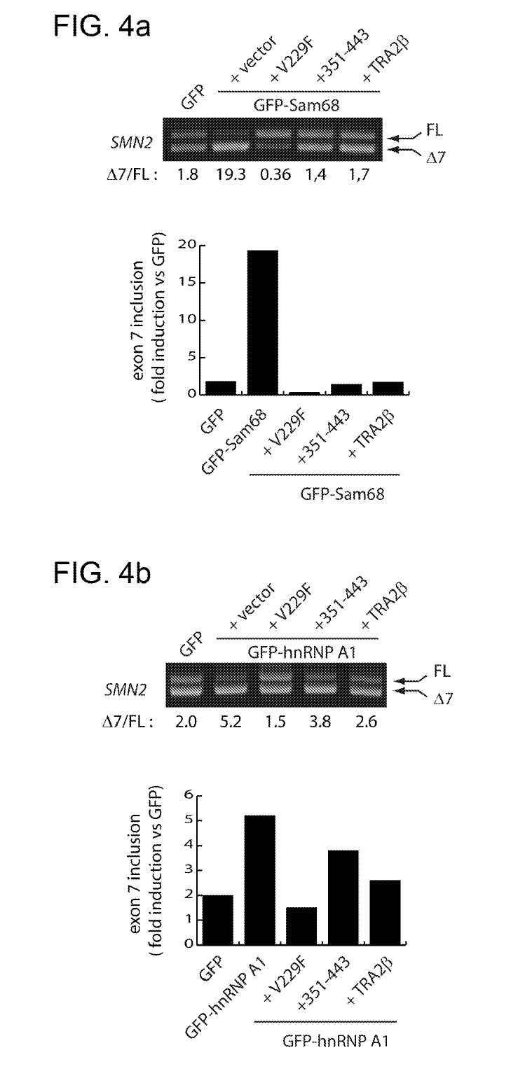

FIG. 4 shows the rescue of exon 7 inclusion in SMN2 in cells transfected with either wild type Sam68 or hnRNP A1.
(A) HEK293T cells were transfected with pCI-SMN2 and a plasmid encoding GFP-Sam68 either alone or with TRA2β, GFP-Sam68_{V229F} or GFP-Sam68₃₅₁₋₄₄₃ plasmids. After 24 hours, cells were analysed by RT-PCR for alternative splicing. Densitometric analysis of the splicing assay is shown below. (B) HEK293T cells were transfected with pCI-SMN2 and a plasmid encoding GFP-hnRNP A1 either alone or with TRA2β GFP-Sam68_{V229F} or GFP-Sam68₃₅₁₋₄₄₃ plasmids. After 24 hours, cells were analysed by RT-PCR for alternative splicing. Densitometric analysis of the splicing assay is shown below.

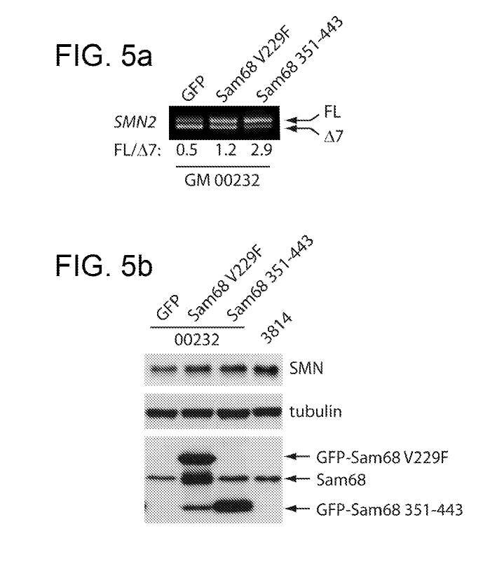

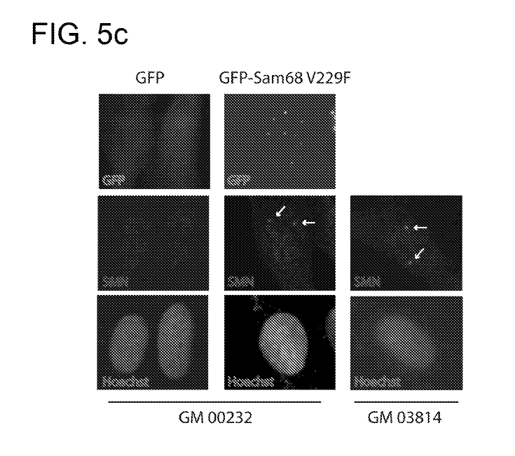

FIG. 5 shows SMN2 protein accumulation and SMN gems in SMA cells due to Sam68 Sam68_{V229F} or GFP-Sam68₃₅₁₋₄₄₃.
(A) Fibroblasts from a SMA patient (GM00232) were infected with retroviruses encoding GFP, GFP-Sam68_{V229F} or GFP-Sam68₃₅₁₋₄₄₃. After selection by sorting for the GFP signal, cells were analysed by RT-PCR for the endogenous SMN2 transcripts. Densitometric analysis is reported below the panel. (B) Western blot analysis of SMN, GFP-fusion proteins and tubulin of the samples analysed in (A). GM03814 wild type fibroblasts are shown as control. (C) Immunofluorescence analysis of SMN in cells analysed in (B). The position of the nuclear gems formed by SMN is indicated by arrows.

### DETAILED DESCRIPTION OF THE INVENTION

According to the present invention a dominant negative mutant of Sam68 is used for the manufacture of a medicament, in particular for the treatment of SMA.

In particular, the dominant negative mutant of Sam68 is used for the manufacture of a medicament for the treatment of SMA so that survival motor neuron (SMN) protein expression is rescued in the cells of an individual affected by SMA.

In one embodiment, the dominant negative mutant of Sam68 comprises at least one amino acid substitution in the region corresponding to amino acids 81 to 276. More preferably, said at least one amino acid substitution is from valine to phenylalanine at position 229.

In another embodiment, the dominant negative mutant of Sam68 comprises at least one amino acid substitution in the region corresponding to amino acids 419 to 443, preferably the dominant negative mutant has an amino acid substitution from arginine to alanine at position 436 and/or an amino acid substitution from arginine to alanine at position 442.

In another embodiment, the dominant negative mutant of Sam68 consists of amino acids 351-443.

In another embodiment, the dominant negative mutant of Sam68 is encoded by a nucleic acid.

In another embodiment the nucleic acid encoding the dominant negative mutant of Sam68 is included in a vector for gene therapy.

Finally, according to the present invention a method for rescuing survival motor neuron (SMN) protein expression in the cells of an individual affected by spinal muscular atrophy for the treatment of SMA comprises administering a dominant negative mutant Sam68 polypeptide and/or nucleic acid to said cells.

The analysis of SMN2 exon 7 sequence highlighted the presence of a binding site for the STAR (signal transduction and activation of RNA) protein Sam68 just upstream of the consensus sequence for hnRNP A1. Sam68 has been recently demonstrated to regulate the alternative splicing of target genes such as CD44 and BCL2L1 (Matter et al., 2002; Paronetto et al., 2007). Moreover, it has been demonstrated that Sam68 and hnRNP A1 physically associate and cooperate in the regulation of BCL2L1 alternative splicing (Paronetto et al., 2007). Herein, it has been investigated whether Sam68 plays a role in the regulation of SMN2 alternative splicing and whether its function requires an association with hnRNP A1. The results indicate that Sam68 strongly triggers SMN2 exon 7 skipping and that interference with its RNA-binding activity or its association with hnRNP A1 in live cells restores exon 7 inclusion and promotes accumulation of a functional SMN protein in SMA patient cells. Thus, Sam68 is a novel regulator of SMN2 alternative splicing that can affect disease severity and represents a valuable target for the therapeutic approach of SMA.

Sam68 Affects the Alternative Splicing of SMN2 exon 7

The C to T transition at position +6 in exon 7 (underlined below) creates a potential binding site for Sam68 (UUUUA) just upstream of the binding site for hnRNP A1 (UAGACA) in the SMN2 pre-mRNA (FIG. 1A). To determine if Sam68 can indeed affect the alternative splicing of SMN2 exon 7, in vivo splicing assays were performed using a minigene that spans the whole alternatively spliced region from exon 6 to exon 8 of human SMN2 (Stoss et al., 2004). Co-transfection of the SMN2 minigene with increasing amounts of GFP-Sam68 triggered a dose-dependent skipping of exon 7 (FIG. 1B). Remarkably, the effect exerted by Sam68 was similar to that obtained with comparable increasing amounts of GFP-hnRNP A1 (FIG. 1C), a known inducer of SMN2 exon 7 skipping (Kashima and Manley, 2003). On the other hand, up-regulation of TRA2β elicited the opposite effect and enhanced exon 7 inclusion (FIG. 1D) whereas ASF/SF2 did not substantially affect alternative splicing of SMN2 (FIG. 1E). To confirm a role for Sam68 in SMN2 alternative splicing, HEK293 were transfected with si-Sam68 dsRNAs to deplete the endogenous protein or with si-Scrambled dsRNAs as a control. Transfection of the SMN2 minigene indicated that downregulation of Sam68 caused an increase in exon 7 inclusion as compared to control cells (FIG. 1F). These results indicate that Sam68 is a splicing factor that can specifically affect SMN2 exon 7 alternative splicing.

### The RNA-Binding Activity of Sam68 is Required for SMN2 Exon 7 Skipping.

Since a putative consensus site for Sam68 is present in the SMN2 pre-mRNA, tests were performed to assess whether the RNA binding activity of Sam68 was required for exon 7 skipping. Two different mutants of Sam68 were used (FIG. 2A): the V229F allele, which carries a point mutation in the GSG RNA-binding domain that strongly impairs the affinity for RNA, and the NLS-KO allele, which contains mutations in the nuclear localization signal (NLS) and physically impairs the ability of Sam68 to affect splicing in the nucleus (Paronetto et al., 2007). As shown in FIG. 2B, both mutations completely suppressed the ability of Sam68 to induce exon 7 skipping, demonstrating that the RNA binding and the nuclear localization of Sam68 are required for this event. To determine whether Sam68 exerted its effect through binding to the UUUUA consensus created by the C-to-T transition in exon 7, the T at positions +4 and +5 was substituted with G (TT-to-GG mutant) to disrupt this potential binding site. In addition, the A at position +7 was substituted with C to disrupt both the hnRNP A1 and Sam68 consensus sites (A-to-C mutant) or the A and C at position +9 and +10 with T and G (AC-to-TG mutant), which should only affect hnRNP Al binding. The mutations were introduced into the SMN2 minigene and tested for their activity in co-transfection experiments. As shown in FIG. 2C, mutation of the potential Sam68 binding site strongly impaired exon 7 skipping and completely suppressed the effect of Sam68 on alternative splicing of SMN2, indicating that this sequence is required for Sam68-induced exon 7 skipping. An even stronger suppression of exon 7 skipping was obtained mutating the A at position +7, which disrupt the consensus for both Sam68 and hnRNP A1. Also in this case, up-regulation of Sam68 was unable to affect alternative splicing of the exon completely suppressed exon skipping and abolished the effect of up-regulation of either splicing factor. On the other hand, when mutations were introduced in the region containing only the hnRNP A1 consensus, Sam68 was still able to induce exon 7 skipping (FIG. 2C). Similar splicing assays were also performed with hnRNP A1. Remarkably, a complementary behaviour of this splicing factor was observed. hnRNP Al up-regulation could strongly induce exon skipping when the Sam68 binding site was mutated, whereas its effect was strongly impaired in the AC-to-TG mutant. However, a complete suppression of exon skipping even in cells overexpressing either Sam68 or hnRNP A1 was achieved only when both consensus sites were mutated by substitution of A at position +7 with C (FIG. 2C). These results strongly indicate that Sam68 and hnRNP A1 bind to close but distinct sites on the SMN2 exon 7 and that both proteins are required for efficient skipping of this exon from the pre-mRNA.

Sam68 and hnRNP A1 Cooperate in SMN2 Exon 7 Skipping.

The experiments shown above demonstrated that binding of Sam68 is required for SMN2 exon 7 skipping and suggested that the concerted action of Sam68 and hnRNP A1 is required for such event. To further investigate on the possible cooperation between Sam68 and hnRNP A1 in SMN2 alternative splicing, the endogenous proteins were depleted by RNAi. When HEK293 cells were transfected with either Sam68 or hnRNP A1 siRNAs, a small but reproducible decrease in SMN2 exon 7 skipping was observed (FIG. 3A). Remarkably, when both proteins were silenced concomitantly, a synergistic effect on exon inclusion was observed (FIG. 3A), suggesting that Sam68 and hnRNP A1 cooperate in the promotion of SMN2 exon 7 skipping.

As an alternative approach to test the cooperation between these splicing regulators, their ability to counteract the action of TRA2β, a positive regulator of SMN2 exon 7 inclusion, was tested. It was observed that co-expression of either Sam68 or hnRNP A1 inhibited TRA2β-induced exon 7 inclusion. However, when Sam68 and hnRNP A1 were co-expressed, a more than additive effect was observed and exon 7 inclusion was almost completely suppressed even in the presence of excess TRA2β (FIG. 3B). These results further indicate that Sam68 and hnRNP A1 cooperate to induce SMN2 exon 7 skipping.

Mutations that Interfere with Sam68 Activity Restore Exon 7 Inclusion in SMN2 Pre-mRNA.

Sam68 functions as a dimer in vivo (Richard 1999) and it interacts with hnRNP A1 through its carboxyterminal 93 amino acids (Paronetto et al., 2007). If Sam68 and hnRNP A1 cooperate in promoting exon 7 skipping, interference of these functions of Sam68 might limit or revert this effect on SMN2 alternative splicing. In line with this hypothesis, it was observed that Sam68_{V229F}, which is defective in RNA binding activity but homodimerizes with the endogenous Sam68, almost completely suppressed exon 7 skipping when overexpressed in HEK293 cells (FIG. 2B), suggesting that it acts as dominant negative of Sam68, i.e. interacts with endogenous Sam68 and sequesters it into non-functional domains. A similar result on exon 7 inclusion was obtained by overexpression of Sam68₃₅₁₋₄₄₃, a truncated nuclear form of Sam68 that contains the hnRNP A1 binding site but lacks the RNA-binding and the homodimerization domain. To determine whether these dominant-negative alleles of Sam68 could attenuate or inhibit SMN2 exon 7 skipping in live cells, they were co-expressed in HEK293 cells together with either wild type GFP-Sam68 or GFP-hnRNP A1. TRA2β was also co-expressed to compare the activity of the mutated Sam68 proteins with that of a physiological inducer of SMN2 exon 7 inclusion. Remarkably, it was found that GFP-Sam68_{V229F} and GFP-Sam68₃₅₁₋₄₄₃ suppressed exon 7 skipping induced by overexpression of Sam68 or, albeit to a lesser extent, hnRNP A1. Moreover, the effect of GFP-Sam68_{V229F} was even stronger than that exerted by up-regulation of Tra2β. causing an almost complete reversion of the alternative splicing and accumulation of the full-length form of SMN2 above basal levels even in the presence of excess Sam68 or hnRNP A1. These experiments suggest that GFP-Sam68_{V229F} and GFP-Sam68₃₅₁₋₄₄₃ are efficient competitors of SMN2 exon 7 skipping in vivo. The disruption of a functional complex between Sam68 (through interference with its RNA-binding activity) and hnRNP A1 (through competition with its interaction with endogenous Sam68) inhibits exon 7 skipping in the SMN2 pre-mRNA.

### GFP-Sam68_{V229F} and GFP-Sam68₃₅₁₋₄₄₃ Restore Exon 7 Inclusion and Allow SMN2 Protein Accumulation in SMA Cells.

To determine whether GFP-Sam68_{V229F} and GFP-Sam68₃₅₁₋₄₄₃ could affect SMN2 alterative splicing in a physiological setting, SMA fibroblasts were infected with retroviral constructs encoding these Sam68 mutants or GFP as control. Infected cells were sorted for GFP signal and RNA and proteins were extracted. Expression of GFP-Sam68_{V229F} and GFP-Sam68₃₅₁₋₄₄₃ enhanced exon 7 inclusion in the endogenous SMN2 pre-mRNA in patient cells as compared to cells infected with GFP alone (FIG. 5A). This effect on alternative splicing resulted in increased SMN protein production (FIG. 5B). Remarkably, the amount of SMN protein produced after expression of GFP-Sam68_{V229F} and GFP-Sam68₃₅₁₋₄₄₃ was comparable to that observed in control fibroblasts from a donor (GM03814). Moreover, expression of GFP-Sam68V229F (FIG. 5C) and GFP-Sam68351-443 (data not shown) could also restore a functional SMN protein, as demonstrated by the formation of gems in the nucleus like in the donor fibroblasts. These experiments demonstrate that disruption of a functional complex between Sam68 and hnRNP A1 by expressing dominant-negative Sam68 mutant proteins restores SMN activity in SMA cells.

It is apparent to the person skilled in the art that modifications may be made to the methods and procedures without departing from the scope of the invention as set forth in the appended claims.

Advantageously, the invention is intended to include dominant negative mutants of Sam68 in the form of cell-permeable peptides interfering with homodimerization or with hnRNP A1 binding. In order to allow cell penetration, the peptides are N-terminally modified as reviewed in Morris et al. 2008, in particular by fusing 11 arginine residues followed by three glycines. In particular, peptides have a length of about 10 amino acids spanning part or the whole of regions from amino acid 163 to amino acid 171, from amino acid 198 to amino acid 227, or from amino acid 351 to amino acid 443.

### Experimental

### Plasmid Constructs

The pCI-SMN2 and pCI-SMN1 wild type minigenes (Lorson C. L. et al, 1999) and pCDNA3-SMN2 wild type and mutant minigenes (Kashima and Manley, 2003) have been previously described. pCDNA3-Tra2β was generously provided by Dr J L Manley (Columbia University, NY). The plasmids encoding GFP-Sam68, GFP-Sam68V229F, GFP-hnRNP A1 and Flag-ASF/SF2 have been previously described (Paronetto et al, 2007). Sam68₃₅₁₋₄₄₃ was amplified by PCR using Pfu polymerase (Stratagene) and pEGFP-C1-Sam68 as template. The amplified cDNA was subcloned into the EcoRI and SalI sites of pEGFP-C1 (Clontech).

### Cell Culture and Transfection

HEK293 (from ATCC) and human SMA cell lines GM03814, GM03813, GM00232 (from Coriell Repositories) were maintained in Dulbecco's modified Eagle's medium (DMEM; Gibco BRL) supplemented with 10% fetal bovine serum (FBS) (BioWhittaker Cambrex Bioscience), penicillin and streptomycin. For transfections, HEK293 cells were plated in 35 mm dishes 1 day before and transfected with 1 μg of DNA (pCI-SMN2 minigene, pEGFP-Sam68wt, pEGFPSam68_{V229F}, pEGFP Sam68₃₅₁₋₄₄₃, pEGFP-Sam68 NLSKO, pEGFP-hnRNP A1, Flag-ASF/SF2, pCDNA3-Tra2β, pCDNA3-SMN2 wild type or mutated minigenes, pEGFP-C1, using lipofectamine 2000 (Invitrogen) according to the manufacturer's instructions. 24 h after transfections, cells were collected for RNA or biochemical analysis (see below). For RNAi, cells at ˜50/60% confluence were transfected with small interfering RNA (siRNAs) (MWG Biotech) using Lipofectamine RNAi MAX and Opti-MEM medium (Invitrogen) according to the manufacture's instructions. Transfections were performed for two consecutive days. Sequences for Sam68 and hnRNP A1 siRNA are (sense strand): 5′-GGAUCUGCAUGUCUUCAUU-3′ (siSam68), 5′-AGCAAGAGAUGGCUAGUGC-3′ (sihnRNP A1). The sequence used as control is: 5′-GUGCUCAAUUGGAUUCUCU-3′.

Extraction of RNA and Proteins from Cultured Cells

Total RNA was extracted from transfected HEK293 cells and human SMA cell lines GM00232, GM03813, GM03814 using cold TRIzol reagent (Invitrogen), according to the manufacturer's instructions. RNA was resuspended in RNAse-free water (Sigma-Aldrich) and immediately frozen at −80° C. for further analysis. For protein extraction, HEK293 cells or SMA fibroblast were resuspended in lysis buffer (100 mM NaCl, 10 mM MgCl2, 30 mM Tris-HCl, pH 7.5, 1 mM dithiothreitol, 10 mM β-glycerophosphate, 0.5 mM NaVO4, protease inhibitor cocktail), supplemented with 0.5% Triton-X-100 and SMA cell lines extracts were also sonicated. The extracts were centrifuged for 10 min at 12,000×g at 4° C. the supernatants were collected and used for Western blot experiment.

### RT-PCR Analysis

RNA (1 μg) from HEK293 transfected cells and human SMA cell lines was used for RT-PCR using M-MLV reverse transcripase (Invitrogen) according to manufacturer's instructions. 10% of the RT reaction was used as template together with the following primers: pCI (forward) 5′-GGTGTCCACTCCCAGTTCAA-3′, T7 5′-TAATACGACTCACTATAGGG-3′, SMN2 Ex6 (forward) 5′-ATAATTCCCCCACCACCTCC-3′ and SMN2 Ex8 (reverse) 5′-GCCTCACCACCGTGCTGG-3′. 25 cycles of amplifications were performed.

### Western Blot Analysis

Cell extracts were diluted in SDS sample buffer and boiled for 5 minutes. Proteins were separated on 10% or 12% SDS-PAGE gels and transferred to Hybond-P membranes (Amersham) as previously described (Paronetto et al., 2007). The following primary antibody (1:1000 dilution) were used: rabbit anti-Sam68 (Santa Cruz Biotechnology), anti-GFP (Molecular Probe, Invitrogen), mouse anti-hnRNPA1, mouse anti-tubulin (Sigma-Aldrich), mouse anti-SMN (Beckton and Dickinson). Secondary anti-mouse or anti-rabbit IgGs conjugated to horseradish peroxidase (Amersham) were incubated with the membranes for 1 h at room temperature at a 1:10000 dilution in PBS or TBS containing 0.1% Tween 20. Immunostained bands were detected by chemiluminescent method (Santa Cruz Biotechnology).

### Immunofluorescence Analysis

Human SMA cell lines GM03814, GM00232 and GM03813 grown on glass coverslips were rinsed in PBS and fixed in 50% methanol-50% acetone for 10 minutes at −20° C. Cells were then rinsed at room temperature in PBS containing 3% BSA and 0.1% Triton-100X for 30 minutes. Primary antibody against SMN protein (Beckton and Dickinson) (diluted 1:150) were added to the coverlips overnight at 4° C. After three washes in PBS, cells were incubated for 1 hour in the dark and at room temperature with the anti-mouse secondary antibody (Alexa fluo) (diluted 1:400) and with Hoechst 3332 (diluited 1:1000) for nuclei staining. Samples were mounted with MOWIOL solution and fluorescence was observed with a 100× objective.

### Retroviral Expression

For retroviral expression, 15 μg of the retroviral vectors (pCLPCX-GFP or -GFP-Sam68(V229F) or -GFP-Sam68(351-443) were co-transfected with 5 μg of an expression plasmid for the vescicular stomatitis virus G protein into SMA cell lines GM00232 or GM03813 gp/bsr by using the calcium phosphate method. 48 hours later, the supernatant containing the retroviral particles was recovered and supplemented with polybrene (4 μg/mL). GM00232 or GM03813 cells (5×10⁵) were infected by incubation with the retroviruses. Briefly, the infection were carried out in three steps: 1) cells were incubated with the retroviruses for 4 h; 2) the supernatant was removed and infection was repeated with fresh viruses for further 4 h; 3) the supernatant was removed and fresh viral preparation was added and infection carried out overnight. At the end, cells were rinsed and 24-48 hours later selected for GFP expression by cell sorting.

### REFERENCES

1. Johnson J M, Castle J, Garrett-Engele P, Kan Z, Loerch P M, Armour C D, Santos R, Schadt E E, Stoughton R, Shoemaker D D. Genome-wide survey of human alternative pre-mRNA splicing with exon junction microarrays. Science. 2003 Dec. 19; 302(5653):2141-4.
2. Black D L. Mechanisms of alternative pre-messenger RNA splicing. Annu Rev Biochem. 2003; 72:291-336.
3. Wang E T, Sandberg R, Luo S, Khrebtukova I, Zhang L, Mayr C, Kingsmore S F, Schroth G P, Burge C B. Alternative isoform regulation in human tissue transcriptomes. Nature. 2008 Nov. 27; 456(7221):470-6.
4. Pan Q, Shai O, Lee L J, Frey B J, Blencowe B J. Deep surveying of alternative splicing complexity in the human transcriptome by high-throughput sequencing. Nat Genet. 2008 December; 40(12):1413-5.
5. Matlin A J, Clark F, Smith C W. Understanding alternative splicing: towards a cellular code. Nat Rev Mol Cell Biol. 2005 May; 6(5):386-98
6. Yu Y, Maroney P A, Denker J A, Zhang X H, Dybkov O, Luhrmann R, Jankowsky E, Chasin L A, Nilsen T W. Dynamic regulation of alternative splicing by silencers that modulate 5′ splice site competition. Cell. 2008 Dec. 26; 135(7):1224-36.
7. Tazi J, Bakkour N, Stamm S. Alternative splicing and disease. Biochim Biophys Acta. 2009 January; 1792(1):14-26.
8. Wang G S, Cooper T A. Splicing in disease: disruption of the splicing code and the decoding machinery. Nat Rev Genet. 2007 October; 8(10):749-61.
9. Krawczak M, Reiss J, Cooper D N. The mutational spectrum of single base-pair substitutions in mRNA splice junctions of human genes: causes and consequences. Hum Genet. 1992 September-October; 90(1-2):41-54.
10. López-Bigas N, Audit B, Ouzounis C, Parra G, Guigó0 R.

Are splicing mutations the most frequent cause of hereditary disease? FEBS Lett. 2005 Mar. 28; 579(9):1900-3
11. Monani U R. Spinal muscular atrophy: a deficiency in a ubiquitous protein; a motor neuron-specific disease. Neuron. 2005 Dec. 22; 48(6):885-96.
12. Pearn J. Classification of spinal muscular atrophies. Lancet. 1980 Apr. 26; 1(8174):919-22.
13. Zhang Z, Lotti F, Dittmar K, Younis I, Wan L, Kasim M, Dreyfuss G. SMN deficiency causes tissue-specific perturbations in the repertoire of snRNAs and widespread defects in splicing. Cell. 2008 May 16; 133(4):585-600.
14. Kariya S, Park G H, Maeno-Hikichi Y, Leykekhman O, Lutz C, Arkovitz M S, Landmesser L T, Monani U R. Reduced SMN protein impairs maturation of the neuromuscular junctions in mouse models of spinal muscular atrophy. Hum Mol Genet. 2008 Aug. 15; 17(16):2552-69. Epub 2008 May 20.
15. Monani U R, Lorson C L, Parsons D W, Prior T W, Androphy E J, Burghes A H, McPherson J D. A single nucleotide difference that alters splicing patterns distinguishes the SMA gene SMN1 from the copy gene SMN2. Hum Mol Genet. 1999 July; 8(7):1177-83.
16. Lorson C L, Hahnen E, Androphy E J, Wirth B. A single nucleotide in the SMN gene regulates splicing and is responsible for spinal muscular atrophy. Proc Natl Acad Sci USA. 1999 May 25; 96(11):6307-11.
17. Cartegni L, Chew S L, Krainer A R. Listening to silence and understanding nonsense: exonic mutations that affect splicing. Nat Rev Genet. 2002 April; 3(4):285-98.
18. Pellizzoni L. Chaperoning ribonucleoprotein biogenesis in health and disease. EMBO Rep. 2007 April; 8(4):340-5.
19. Cartegni L, Krainer A R. Disruption of an SF2/ASF-dependent exonic splicing enhancer in SMN2 causes spinal muscular atrophy in the absence of SMN1. Nat Genet. 2002 April; 30(4):377-84.
20. Kashima T, Manley J L. A negative element in SMN2 exon 7 inhibits splicing in spinal muscular atrophy. Nat Genet. 2003 August; 34(4):460-3.
21. Kashima T, Rao N, David C J, Manley J L. hnRNP A1 functions with specificity in repression of SMN2 exon 7 splicing. Hum Mol Genet. 2007 Dec. 15; 16(24):3149-59.
22. Hofmann Y, Lorson C L, Stamm S, Androphy E J, Wirth B. Htra2-beta 1 stimulates an exonic splicing enhancer and can restore full-length SMN expression to survival motor neuron 2 (SMN2). Proc Natl Acad Sci USA. 2000 Aug. 15; 97(17):9618-23.
23. Lefebvre S, Burlet P, Liu Q, Bertrandy S, Clermont O, Munnich A, Dreyfuss G, Melki J. Correlation between severity and SMN protein level in spinal muscular atrophy. Nat Genet. 1997 July; 16(3):265-9.
24. Singh R, Valcárcel J. Building specificity with nonspecific RNA-binding proteins. Nat Struct Mol Biol. 2005 August; 12(8):645-53.
25. Chen H H, Chang J G, Lu R M, Peng T Y, Tarn W Y. The RNA binding protein hnRNP Q modulates the utilization of exon 7 in the survival motor neuron 2 (SMN2) gene. Mol Cell Biol. 2008 November; 28(22):6929-38.
26. Bose J K, Wang I F, Hung L, Tarn W Y, Shen C K. TDP-43 overexpression enhances exon 7 inclusion during the survival of motor neuron pre-mRNA splicing. J Biol Chem. 2008 Oct. 24; 283(43):28852-9.
27. Lukong K E, Richard S. Sam68, the KH domain-containing superSTAR. Biochim Biophys Acta. 2003 Dec. 5; 1653(2):73-86.
28. Matter, N., P. Herrlich, and H. Konig. Signal-dependent regulation of splicing via phosphorylation of Sam68. Nature. 2002 420: 691-695.
29. Paronetto M P, Achsel T, Massiello A, Chalfant C E, Sette C. The RNA-binding protein Sam68 modulates the alternative splicing of Bcl-x. J Cell Biol. 2007 176:929-939.
30. Chawla G, Lin C H, Han A, Shiue L, Ares M Jr, Black D L. Sam68 regulates a set of alternatively spliced exons during neurogenesis. Mol Cell Biol. 2009 January; 29(1):201-13.
31. Li, J., Liu, Y., Kim, B. O., He, J. J. (2002) Direct participation of Sam68, the 68-kilodalton Src-associated protein in mitosis, in the CRM1-mediated Rev nuclear export pathway. J Virol. 76, 8374-8382.
32. Coyle, J. H., Guzik, B. W., Bor, Y. C., Jin, L., Eisner-Smerage, L., Taylor, S. J., Rekosh, D., and Hammarskjold, M. L. (2003). Sam68 enhances the cytoplasmic utilization of intron-containing RNA and is functionally regulated by the nuclear kinase Sik/BRK. Mol Cell Biol. 23, 92-103.
33. Paronetto, M. P., Zalfa, F., Botti, F., Geremia, R., Bagni, C., and Sette, C., (2006). The nuclear RNA-binding protein Sam68 translocates to the cytoplasm and associates with the polysomes in mouse spermatocytes. Mol. Biol. Cell 17, 14-24.
34. Grange J, Belly A, Dupas S, Trembleau A, Sadoul R, Goldberg Y. Specific interaction between Sam68 and neuronal mRNAs: implication for the activity-dependent biosynthesis of elongation factor eEF1A. J Neurosci Res. 2009 January; 87(1):12-25.
35. Lin Q, Taylor S J, Shalloway D. Specificity and determinants of Sam68 RNA binding. Implications for the biological function of K homology domains. J Biol Chem. 1997 Oct. 24; 272(43):27274-80.
36. Di Fruscio M, Chen T, Richard S. Characterization of Sam68-like mammalian proteins SLM-1 and SLM-2: SLM-1 is a Src substrate during mitosis. Proc Natl Acad Sci USA. 1999 Mar. 16; 96(6):2710-5.
37. Kolb S J, Battle D J, Dreyfuss G. Molecular functions of the SMN complex. J Child Neurol. 2007 August; 22(8):990-4.
38. Hua Y, Vickers T A, Baker B F, Bennett C F, Krainer A R. Enhancement of SMN2 exon 7 inclusion by antisense oligonucleotides targeting the exon. PLoS Biol. 2007 April; 5(4):e73.
39. Hua Y, Vickers T A, Okunola H L, Bennett C F, Krainer A R. Antisense masking of an hnRNP A1/A2 intronic splicing silencer corrects SMN2 splicing in transgenic mice. Am J Hum Genet. 2008 April; 82(4):834-48.
40. Madocsai C, Lim S R, Geib T, Lam B J, Hertel K J. Correction of SMN2 Pre-mRNA splicing by antisense U7 small nuclear RNAs. Mol Ther. 2005 December; 12(6):1013-22.
41. Baughan T, Shababi M, Coady T H, Dickson A M, Tullis G E, Lorson C L. Stimulating full-length SMN2 expression by delivering bifunctional RNAs via a viral vector. Mol Ther. 2006 July; 14(1):54-62.
42. Marquis J, Meyer K, Angehrn L, Kämpfer S S, Rothen-Rutishauser B, Schümperli D. Spinal muscular atrophy: SMN2 pre-mRNA splicing corrected by a U7 snRNA derivative carrying a splicing enhancer sequence. Mol Ther. 2007 August; 15(8):1479-86.
43. Coady T H, Baughan T D, Shababi M, Passini M A, Lorson C L. Development of a single vector system that enhances trans-splicing of SMN2 transcripts. PLoS ONE. 2008; 3(10):e3468.
44. DiMatteo D, Callahan S, Kmiec E B. Genetic conversion of an SMN2 gene to SMN1: a novel approach to the treatment of spinal muscular atrophy. Exp Cell Res. 2008 Feb. 15; 314(4):878-86.
45. Novoyatleva T, Heinrich B, Tang Y, Benderska N, Butchbach M E, Lorson C L, Lorson M A, Ben-Dov C, Fehlbaum P, Bracco L, Burghes A H, Bollen M, Stamm S. Protein phosphatase 1 binds to the RNA recognition motif of several splicing factors and regulates alternative pre-mRNA processing. Hum Mol Genet. 2008 Jan. 1; 17(1):52-70.
46. Angelozzi C, Borgo F, Tiziano F D, Martella A, Neri G, Brahe C. Salbutamol increases SMN mRNA and protein levels in spinal muscular atrophy cells. J Med Genet. 2008 January; 45(1):29-31.
47. Kinali M, Mercuri E, Main M, De Biasia F, Karatza A, Higgins R, Banks L M, Manzur A Y, Muntoni F. Pilot trial of albuterol in spinal muscular atrophy. Neurology. 2002 Aug. 27; 59(4):609-10.
48. Pane M, Staccioli S, Messina S, D'Amico A, Pelliccioni M, Mazzone E S, Cuttini M, Alfieri P, Battini R, Main M, Muntoni F, Bertini E, Villanova M, Mercuri E. Daily salbutamol in young patients with SMA type II. Neuromuscul Disord. 2008 July; 18(7):536-40.
49. Rajan P, Gaughan L, Dalgliesh C, El-Sherif A, Robson C N, Leung H Y, Elliott D J. The RNA-binding and adaptor protein Sam68 modulates signal-dependent splicing and transcriptional activity of the androgen receptor. J Pathol. 2008 May; 215(1):67-77.
50. Paronetto M P, Venables J P, Elliott D J, Geremia R, Rossi P, Sette C. Tr-kit promotes the formation of a multimolecular complex composed by Fyn, PLCgammal and Sam68. Oncogene. 2003 Nov. 27; 22(54):8707-15.
51. Tsai L K, Tsai M S, Ting C H, Wang S H, Li H. Restoring Bcl-x(L) levels benefits a mouse model of spinal muscular atrophy. Neurobiol Dis. 2008 September; 31(3):361-7.
52. Schumacher B, Hanazawa M, Lee M H, Nayak S, Volkmann K, Hofmann E R, Hengartner M, Schedl T, Gartner A. Translational repression of C. elegans p53 by GLD-1 regulates DNA damage-induced apoptosis. Cell. 2005 Feb. 11; 120(3):357-68.
53. Morris C M, Deshayes S, Heitz F, Divita G. Cell-penetrating peptides: from molecular mechanisms to therapeutics. 2008 April; 100(4): 201-217

1. (canceled)

2. The method of claim 14, such that survival motor neuron (SMN) protein expression is rescued in the cells of an individual affected by SMA.

3. The method of claim 14, characterized in that said dominant negative mutant of SEQ. ID. NO:1 comprises at least one amino acid substitution in the region corresponding to amino acids 81 to 276.

4. The method of claim 3, characterized in that said at least one amino acid substitution is from valine to phenylalanine at position 229.

5. The method of claim 14, characterized in that said dominant negative mutant of SEQ. ID. NO:1 comprises at least one amino acid substitution in the region corresponding to amino acids 419 to 443.

6. The method of claim 5, characterized in that said dominant negative mutant of SEQ. ID. NO:1 has an amino acid substitution from arginine to alanine at position 436.

7. The method of claim 5, characterised in that said dominant negative mutant of SEQ. ID. NO:1 has an amino acid substitution from arginine to alanine at position 442.

8. The method of claim 14, wherein said dominant negative mutant is a polypeptide of SEQ ID NO:4.

9. The method of claim 14, wherein said dominant negative mutant of SEQ. ID. NO:1 is encoded by a nucleic acid.

10. A vector for gene therapy including a nucleic acid encoding for a dominant negative mutant of SEQ ID NO:1.

11. A dominant negative mutant of SEQ. ID. NO:1 for use in the treatment of SMA.

12. A method for rescuing survival motor neuron (SMN) protein expression in cells of an individual affected by spinal muscular atrophy for the treatment of SMA comprising administering a dominant negative mutant Sam68 polypeptide and/or nucleic acid to said cells.

13. The method of claim 12, wherein said dominant negative mutant of SEQ ID NO:1 is a dominant negative mutant according to claim 14.

14. A method of treating SMA comprising use of a dominant negative mutant of SEQ. ID. NO:1.


SEQUENCE LISTING (8 sequences)

SEQ ID NO: 1 - protein, 443 aa, Homo Sapiens
MQRRDDPAARMSRSSGRSGSMDPSGAHPSVRQTPSRQPPLPHRSRGGGGGSRGGARASPATQPPPLLPPSATGPDATVGGPAPTPLLPPSATASVKMEPENKYLPELMAEKDSLDPSFTHAMQLLTAEIEKIQKGDSKKDDEENYLDLFSHKNMKLKERVLIPVKQYPKFNFVGKILGPQGNTIKRLQEETGAKISVLGKGSMRDKAKEEELRKGGDPKYAHLNMDLHVFIEVFGPPCEAYALMAHAMEEVKKFLVPDMMDDICQEQFLELSYLNGVPEPSRGRGVPVRGRGAAPPPPPVPRGRGVGPPRGALVRGTPVRGAITRGATVTRGVPPPPTVRGAPAPRARTAGIQRIPLPPPPAPETYEEYGYDDTYAEQSYEGYEGYYSQSQGDSEYYDYGHGEVQDSYEAYGQDDWNGTRPSLKAPPARPVKGAYREHPYGRY

SEQ ID NO: 2 - protein, 443 aa, Homo Sapiens
MQRRDDPAARMSRSSGRSGSMDPSGAHPSVRQTPSRQPPLPHRSRGGGGGSRGGARASPATQPPPLLPPSATGPDATVGGPAPTPLLPPSATASVKMEPENKYLPELMAEKDSLDPSFTHAMQLLTAEIEKIQKGDSKKDDEENYLDLFSHKNMKLKERVLIPVKQYPKFNFVGKILGPQGNTIKRLQEETGAKISVLGKGSMRDKAKEEELRKGGDPKYAHLNMDLHFFIEVFGPPCEAYALMAHAMEEVKKFLVPDMMDDICQEQFLELSYLNGVPEPSRGRGVPVRGRGAAPPPPPVPRGRGVGPPRGALVRGTPVRGAITRGATVTRGVPPPPTVRGAPAPRARTAGIQRIPLPPPPAPETYEEYGYDDTYAEQSYEGYEGYYSQSQGDSEYYDYGHGEVQDSYEAYGQDDWNGTRPSLKAPPARPVKGAYREHPYGRY

SEQ ID NO: 3 - protein, 443 aa, Homo Sapiens
MQRRDDPAARMSRSSGRSGSMDPSGAHPSVRQTPSRQPPLPHRSRGGGGGSRGGARASPATQPPPLLPPSATGPDATVGGPAPTPLLPPSATASVKMEPENKYLPELMAEKDSLDPSFTHAMQLLTAEIEKIQKGDSKKDDEENYLDLFSHKNMKLKERVLIPVKQYPKFNFVGKILGPQGNTIKRLQEETGAKISVLGKGSMRDKAKEEELRKGGDPKYAHLNMDLHVFIEVFGPPCEAYALMAHAMEEVKKFLVPDMMDDICQEQFLELSYLNGVPEPSRGRGVPVRGRGAAPPPPPVPRGRGVGPPRGALVRGTPVRGAITRGATVTRGVPPPPTVRGAPAPRARTAGIQRIPLPPPPAPETYEEYGYDDTYAEQSYEGYEGYYSQSQGDSEYYDYGHGEVQDSYEAYGQDDWNGTRPSLKAPPARPVKGAYAEHPYGAY

SEQ ID NO: 4 - protein, 93 aa, Homo Sapiens
GIQRIPLPPPPAPETYEEYGYDDTYAEQSYEGYEGYYSQSQGDSEYYDYGHGEVQDSYEAYGQDDWNGTRPSLKAPPARPVKGAYREHPYGRY

SEQ ID NO: 5 - DNA, 1332 bp, Homo Sapiens
atgcagcgccgggacgaccccgccgcgcgcatgagccggtcttcgggccgtagcggctccatggacccctccggtgcccacccctcggtgcgtcagacgccgtctcggcagccgccgctgcctcaccggtcccggggaggcggagggggatcccgcgggggcgcccgggcctcgcccgccacgcagccgccaccgctgctgccgccctcggccacgggtcccgacgcgacagtgggcgggccagcgccgaccccgctgctgcccccctcggccacagcctcggtcaagatggagccagagaacaagtacctgcccgaactcatggccgagaaggactcgctcgacccgtccttcactcacgccatgcagctgctgacggcagaaattgagaagattcagaaaggagactcaaaaaaggatgatgaggagaattacttggatttattttctcataagaacatgaaactgaaagagcgagtgctgatacctgtcaagcagtatcccaagttcaattttgtggggaagattcttggaccacaagggaatacaatcaaaagactgcaggaagagactggtgcaaagatctctgtattgggaaagggctcaatgagagacaaagccaaggaggaagagctgcgcaaaggtggagaccccaaatatgcccacttgaatatggatctgcatgtcttcattgaagtctttggacccccatgtgaggcttatgctcttatggcccatgccatggaggaagtcaagaaatttctagtaccggatatgatggatgatatctgtcaggagcaatttctagagctgtcctacttgaatggagtacctgaaccctctcgtggacgtggggtgccagtgagaggccggggagctgcacctcctccaccacctgttcccaggggccgtggtgttggaccacctcggggggctttggtacgtggtacaccagtaaggggagccatcaccagaggtgccactgtgactcgaggcgtgccacccccacctactgtgaggggtgctccagcaccaagagcacggacagcgggcatccagaggatacctttgcctccacctcctgcaccagaaacatatgaagaatatggatatgatgatacatacgcagaacaaagttacgaaggctacgaaggctattacagccagagtcaaggggactcagaatattatgactatggacatggggaggttcaagattcttatgaagcttatggccaggacgactggaatgggaccaggccgtcgctgaaggcccctcctgctaggccagtgaagggagcatacagagagcacccatatggacgttattaa

SEQ ID NO: 6 - DNA, 1332 bp, Homo Sapiens
atgcagcgccgggacgaccccgccgcgcgcatgagccggtcttcgggccgtagcggctccatggacccctccggtgcccacccctcggtgcgtcagacgccgtctcggcagccgccgctgcctcaccggtcccggggaggcggagggggatcccgcgggggcgcccgggcctcgcccgccacgcagccgccaccgctgctgccgccctcggccacgggtcccgacgcgacagtgggcgggccagcgccgaccccgctgctgcccccctcggccacagcctcggtcaagatggagccagagaacaagtacctgcccgaactcatggccgagaaggactcgctcgacccgtccttcactcacgccatgcagctgctgacggcagaaattgagaagattcagaaaggagactcaaaaaaggatgatgaggagaattacttggatttattttctcataagaacatgaaactgaaagagcgagtgctgatacctgtcaagcagtatcccaagttcaattttgtggggaagattcttggaccacaagggaatacaatcaaaagactgcaggaagagactggtgcaaagatctctgtattgggaaagggctcaatgagagacaaagccaaggaggaagagctgcgcaaaggtggagaccccaaatatgcccacttgaatatggatctgcatttcttcattgaagtctttggacccccatgtgaggcttatgctcttatggcccatgccatggaggaagtcaagaaatttctagtaccggatatgatggatgatatctgtcaggagcaatttctagagctgtcctacttgaatggagtacctgaaccctctcgtggacgtggggtgccagtgagaggccggggagctgcacctcctccaccacctgttcccaggggccgtggtgttggaccacctcggggggctttggtacgtggtacaccagtaaggggagccatcaccagaggtgccactgtgactcgaggcgtgccacccccacctactgtgaggggtgctccagcaccaagagcacggacagcgggcatccagaggatacctttgcctccacctcctgcaccagaaacatatgaagaatatggatatgatgatacatacgcagaacaaagttacgaaggctacgaaggctattacagccagagtcaaggggactcagaatattatgactatggacatggggaggttcaagattcttatgaagcttatggccaggacgactggaatgggaccaggccgtcgctgaaggcccctcctgctaggccagtgaagggagcatacagagagcacccatatggacgttattaa

SEQ ID NO: 7 - DNA, 1332 bp, Homo Sapiens
atgcagcgccgggacgaccccgccgcgcgcatgagccggtcttcgggccgtagcggctccatggacccctccggtgcccacccctcggtgcgtcagacgccgtctcggcagccgccgctgcctcaccggtcccggggaggcggagggggatcccgcgggggcgcccgggcctcgcccgccacgcagccgccaccgctgctgccgccctcggccacgggtcccgacgcgacagtgggcgggccagcgccgaccccgctgctgcccccctcggccacagcctcggtcaagatggagccagagaacaagtacctgcccgaactcatggccgagaaggactcgctcgacccgtccttcactcacgccatgcagctgctgacggcagaaattgagaagattcagaaaggagactcaaaaaaggatgatgaggagaattacttggatttattttctcataagaacatgaaactgaaagagcgagtgctgatacctgtcaagcagtatcccaagttcaattttgtggggaagattcttggaccacaagggaatacaatcaaaagactgcaggaagagactggtgcaaagatctctgtattgggaaagggctcaatgagagacaaagccaaggaggaagagctgcgcaaaggtggagaccccaaatatgcccacttgaatatggatctgcatgtcttcattgaagtctttggacccccatgtgaggcttatgctcttatggcccatgccatggaggaagtcaagaaatttctagtaccggatatgatggatgatatctgtcaggagcaatttctagagctgtcctacttgaatggagtacctgaaccctctcgtggacgtggggtgccagtgagaggccggggagctgcacctcctccaccacctgttcccaggggccgtggtgttggaccacctcggggggctttggtacgtggtacaccagtaaggggagccatcaccagaggtgccactgtgactcgaggcgtgccacccccacctactgtgaggggtgctccagcaccaagagcacggacagcgggcatccagaggatacctttgcctccacctcctgcaccagaaacatatgaagaatatggatatgatgatacatacgcagaacaaagttacgaaggctacgaaggctattacagccagagtcaaggggactcagaatattatgactatggacatggggaggttcaagattcttatgaagcttatggccaggacgactggaatgggaccaggccgtcgctgaaggcccctcctgctaggccagtgaagggagcatacgcagagcacccatatggagcttattaa

SEQ ID NO: 8 - DNA, 282 bp, Homo Sapiens
ggcatccagaggatacctttgcctccacctcctgcaccagaaacatatgaagaatatggatatgatgatacatacgcagaacaaagttacgaaggctacgaaggctattacagccagagtcaaggggactcagaatattatgactatggacatggggaggttcaagattcttatgaagcttatggccaggacgactggaatgggaccaggccgtcgctgaaggcccctcctgctaggccagtgaagggagcatacagagagcacccatatggacgttattaa